(19) United States
(12) Patent Application Publication
(10) Pub. No.: US 2017/0197006 A1
(43) Pub. Date: Jul. 13, 2017

(54) HONEY-BASED FOAM COMPOSITIONS

(71) Applicant: Derma Sciences, Inc., Princeton, NJ (US)

(72) Inventors: Keith A. JOHNSON, Durham, NC (US); Rehan N. KHANZADA, Columbus, NJ (US); Mario B.f. NETO, Chester Springs, PA (US); Barry J. WOLFENSON, New Hope, PA (US)

(73) Assignee: Derma Sciences, Inc., Princeton, NJ (US)

(21) Appl. No.: 15/325,000

(22) PCT Filed: Jul. 9, 2015
(86) PCT No.: PCT/US2015/039793

§ 371 Date: Jan. 9, 2017

Related U.S. Application Data
(60) Provisional application No. 62/022,570, filed on Jul. 9, 2014.
(60) Provisional application No. 62/108,448, filed on Jan. 27, 2015.

(51) Int. Cl. A61L 15/42; A61L 15/40; A61L 15/48; A61F 13/00; A61L 15/60; A61M 35/00; A61F 13/02; A61L 15/44; A61L 15/26

(52) U.S. Cl. CPC A61L 15/425; A61L 15/44; A61L 15/40; A61L 15/48; A61L 15/26; A61L 15/60; A61M 35/00; A61F 13/0203; A61F 13/00063; A61F 13/0266; A61L 2300/30; A61L 2300/404

(57) ABSTRACT

Provided are compositions for wound treatment comprising foam impregnated with honey, methods of making such compositions, and methods of treating wounds with such compositions.

### CROSS-REFERENCE TO RELATED APPLICATIONS

This application claims priority from U.S. Provisional Application No. 62/022,570, filed Jul. 9, 2014, and U.S. Provisional Application No. 62/108,448, filed Jan. 27, 2015, the contents of each of which are incorporated by reference herein in their entirety.

### TECHNICAL FIELD

Provided herein are compositions for wound treatment comprising foam impregnated with honey, methods of making such compositions, and methods of treating wounds using such compositions.

### BACKGROUND

Honey has long been used in treatment of wounds. Honey has been shown to reduce symptoms such as inflammation, swelling, and pain, allow for sloughing of necrotic tissue without the need for debridement, and stimulate growth of tissues to repair the wound. Honey has also been shown to have antimicrobial properties that may be important in facilitating wound healing.

One of the difficulties of using honey in wound dressing applications is that it is a naturally sticky substance that is fluid and runny at room temperature. This makes honey difficult to apply and difficult to localize to the desired area. Accordingly, it is desirable to develop wound dressings in which honey is contained within the dressing material, rather than simply applied to the surface of the wound dressing and not being impregnated into the dressing's structures. Furthermore, different types of dressing are suitable for different types of wounds, and thus it is desirable to develop honey-containing versions of many different types of wound dressings.

Currently, dressings such as alginate wound dressings containing honey and gauze wound dressings containing honey are available. However, there are disadvantages to these types of dressings in certain applications. For instance, gauze dressings may stick to a wound, are not recommended for moist wound treatment, and need to be changed frequently. Alginate dressings also need to be changed frequently, and they may adhere to wounds with little or no exudate, causing pain and damaging healthy tissue on removal.

Foam dressings have certain advantages in wound treatment. They are non-adherent, non-linting, and are easy to apply and remove. They can transmit moisture vapor and oxygen and provide thermal insulation to the wound bed. They are suitable for wounds heavily leaking fluids, as they can absorb light to heavy amounts of exudate and facilitate uniform dispersion of exudate throughout the absorbent layer. The high absorbency rate of foam may also allow for an increase in the time between dressing changes. Despite the advantages of foam dressings in certain applications, it has been a challenge to achieve impregnation of foam dressings with a therapeutically effective amount of honey, and thus foam dressings impregnated with honey are not currently available. Thus, there is a need for honey-impregnated foam dressings for wound treatment and suitable methods for preparing such dressings.

### BRIEF SUMMARY OF THE INVENTION

In one aspect, provided herein is a composition for treating wounds, comprising foam impregnated with honey and a surfactant. In some embodiments, the surfactant is a carbohydrate-based surfactant. In some embodiments, the surfactant is a glucoside surfactant.

In some embodiments, the honey contains some added water.

In some embodiments, the honey contains some added water and a surfactant.

In some embodiments, the honey has antimicrobial or bacteriostatic properties. In some embodiments, the honey has non-peroxide antimicrobial or bacteriostatic properties.

In some embodiments, the composition further comprises a tackiness-reducing agent. In some embodiments, the tackiness-reducing agent is carboxymethylcellulose (CMC).

In some embodiments, the composition further comprises a backing sheet. In some embodiments, the backing sheet is adhesive.

In some embodiments, the foam has about 40 to about 100 pores per inch (PPI).

In some embodiments, the foam is a polyurethane foam.

In some embodiments, the composition further comprises beta-glucan or other hydrocolloid.

In some embodiments, the composition is impregnated with a therapeutically effective amount of the honey.

In some embodiments, the composition further comprises a super-absorbent polymer laminated to the foam.

In some embodiments, the composition further comprises a peelable cover sheet adhered to the dressing.

In some embodiments, the composition is sterile.

In some embodiments, the foam is a felted foam.

In some embodiments, the foam is a patterned foam.

In another aspect, provided herein is a composition for treating wounds, comprising foam impregnated with honey at a level of at least about 0.5 grams per square inch. In some embodiments, the foam dressing is impregnated with honey at a level of at least about 0.9 grams per square inch. In some embodiments, the foam dressing is impregnated with honey at a level of at least about 1.6 grams per square inch.

In some embodiments, the composition further comprises a surfactant. In some embodiments, the surfactant is a carbohydrate-based surfactant. In some embodiments, the surfactant is a glucoside surfactant.

In some embodiments, the honey has antimicrobial or bacteriostatic properties. In some embodiments, the honey has non-peroxide antimicrobial or bacteriostatic properties.

In some embodiments, the composition further comprises a tackiness-reducing agent. In some embodiments, the tackiness-reducing agent is carboxymethylcellulose (CMC).

In some embodiments, the composition further comprises a backing sheet. In some embodiments, the backing sheet is adhesive.

In some embodiments, the foam has about 40 to about 100 pores per inch (PPI).

In some embodiments, the foam is a polyurethane foam.

In some embodiments, the composition further comprises beta-glucan or other hydrocolloid.

In some embodiments, the composition further comprises a super-absorbent polymer laminated to the foam.

In some embodiments, the composition further comprises a peelable cover sheet adhered to the dressing.

In some embodiments, the composition is sterile.

In some embodiments, the foam is a felted foam.

In some embodiments, the foam is a patterned foam.

In another aspect, provided herein is a method of making a wound dressing, comprising a) mixing honey, water, and a surfactant; and b) applying the mixture of step a) to the surface of a piece of foam.

In some embodiments, the mixture of step a) comprises at least about 50% honey. In some embodiments, the mixture of step a) comprises less than about 50% water. In some embodiments, the mixture of step a) comprises about 0.5 to about 5% surfactant. In some embodiments, the mixture of step a) comprises about 80 to about 94% honey, about 5 to about 15% water, and about 1 to about 5% surfactant.

In some embodiments, one or both of steps a) and b) are carried out at room temperature. In some embodiments, one or both of steps a) and b) are carried out such that the honey reaches a temperature between about 35 and about 45° C. at some time during the process.

In some embodiments, the method further comprises drying the foam after step b) to reduce the water content.

In some embodiments, the method further comprises applying a tackiness-reducing agent to the foam after step b). In some embodiments, the tackiness-reducing agent is carboxymethylcellulose (CMC).

In another aspect, provided herein is a method of treating a wound in an individual, comprising applying a composition comprising foam impregnated with honey and a surfactant thereto. In some embodiments, the wound is selected from the group consisting of a skin ulcer, a leg ulcer, a diabetic foot ulcer, a pressure ulcer, a sutured wound, a skin graft, a burn, a wound requiring negative pressure wound therapy, a surgical wound, an oral wound, and an exuding wound.

In another aspect, provided herein is a method of treating a wound in an individual, comprising applying a composition comprising foam impregnated with honey at a level of at least about 0.5 grams per square inch thereto. In some embodiments, the wound is selected from the group consisting of a skin ulcer, a leg ulcer, a diabetic foot ulcer, a pressure ulcer, a sutured wound, a skin graft, a burn, a wound requiring negative pressure wound therapy, a surgical wound, an oral wound, and an exuding wound.

### BRIEF DESCRIPTION OF THE DRAWINGS

FIG. 1 is a side view of an exemplary wound dressing.

FIG. 2 is a side view of an exemplary wound dressing.

FIG. 3 is a contour plot of rate of honey impregnation into a polyurethane foam as a function of percentage of water and surfactant in the honey formulation.

FIG. 4 illustrates an exemplary patterned foam dressing.

FIG. 5 illustrates an exemplary patterned foam dressing.

FIG. 6 illustrates an exemplary patterned foam dressing.

### DETAILED DESCRIPTION OF THE INVENTION

Impregnating foam with honey has been a technical challenge. A variety of methods for impregnating foam with honey have been attempted, with limited success. Neat honey applied the surface of a foam dressing may not substantially penetrate the foam dressing at a suitable rate for production. Brayers, rollers, and laminators can be used to apply mild to moderate pressure to neat honey on the surface of a foam dressing; however, the honey may flow along the foam's surface and not substantially penetrate the foam dressing, either at room temperature or at temperatures of 38-42° C. Vibration and heat may not improve penetration of the honey into the foam dressing. The hardness and wettability (i.e., hydrophobicity or hydrophilicity) of the roller may also have little to no effect.

The present inventors have developed a method for consistently and reliably impregnating a wide variety of foams with honey. The method can be applied to individual foam dressings, or it can be used in large-scale manufacturing in an automated process to impregnate extensive sheets of foam with honey. Accordingly, provided herein are compositions comprising foam impregnated with honey, methods for making such compositions, and methods of treating wounds using such compositions.

As used herein, the terms “including,” “containing,” and “comprising” are used in their open, non-limiting sense.

As used herein, the terms “a,” “an,” and “the” refer to one or more, unless otherwise indicated.

It is to be understood that numerical values provided herein as a percentage (e.g., 50%) refer to % w/w, unless otherwise indicated.

As used herein, the term “foam” refers to a dispersion of air in a solid. Foams can be made from any suitable material including, without limitation, polyurethane, polyvinyl alcohol, and silicone. Foams can be homogenous or heterogeneous, and they may be composed of a single material or multiple different materials. The foam may be in any suitable form including, without limitation, pads, sheets, and pillow (cavity) dressings. The foam may have any suitable shape, size, area, thickness, or density.

As used herein, the term “carbohydrate-based surfactant” refers to an amphiphilic compound containing both a saccharide and a hydrophobic tail. The surfactant may be naturally occurring or synthetic. The surfactant may contain one or more saccharides and one or more hydrophobic tails. The saccharide portion of the surfactant may be derived from any naturally-occurring or synthetic mono-, di-, or poly-saccharide including, without limitation, glucose, mannose, allose, altrose, mannose, gulose, iodose, galactose, talose, ribose, arabinose, xylose, lyxose, pyranose, dextrose, and sucrose. The hydrophobic tail portion of the surfactant may be derived from any naturally-occurring or synthetic molecule. The hydrophobic tail may contain, without limitation, a linear or branched, saturated or unsaturated alkyl group or fatty alcohol group. Exemplary hydrophobic tails contain C₆-C₂₀ alkyl or C₆-C₂₀ fatty alcohol groups.

As used herein, the term “impregnated” and its derivatives (e.g., “impregnate”) refers to a state of being infused or permeated with a given substance. A material impregnated with a given substance may be partially or fully saturated with the substance. The degree of impregnation may be uniform or heterogeneous throughout a given material.

As used herein, the term “therapeutically effective amount” refers to an amount of a therapeutic agent necessary to achieve a desired physiologic effect (e.g., to treat wounds).

As used herein, the term “treat” and its derivatives (e.g., “treatment,” “treating”) refers to an approach for obtaining a beneficial or desired result, such as a clinical result. Beneficial or desired clinical results include, without limitation, alleviation of a symptom and/or diminishment of the extent of a symptom and/or preventing a worsening of a symptom associated with a disease or condition. In one variation, beneficial or desired clinical results include, without limitation, alleviation of a symptom and/or diminishment of the extent of a symptom and/or preventing a worsening of a symptom associated with a wound. Symptoms which may be alleviated, diminished, or prevented include, without limitation, pain, tenderness, bleeding, exudation, redness, swelling, heat, fever, inability to use or move the affected area, foul smell, skin opening, oozing, inflammation, and pus. Preferably, treatment of a disease or condition with a composition described herein is accompanied by no or fewer side effects than are associated with currently available therapies for the disease or condition and/or improves the quality of life of the individual.

As used herein, and unless otherwise indicated, the term “individual” refers to a mammal, including but not limited to a human. An individual includes but is not limited to human, bovine, primate, equine, canine, feline, porcine, and ovine animals. Thus, the compositions and methods described herein find use in both human medicine and in the veterinary context, including use in agricultural animals and domestic pets.

As used herein, the term “pharmaceutically acceptable” means that the components of the composition are suitable for administration to the individual being treated.

### Compositions

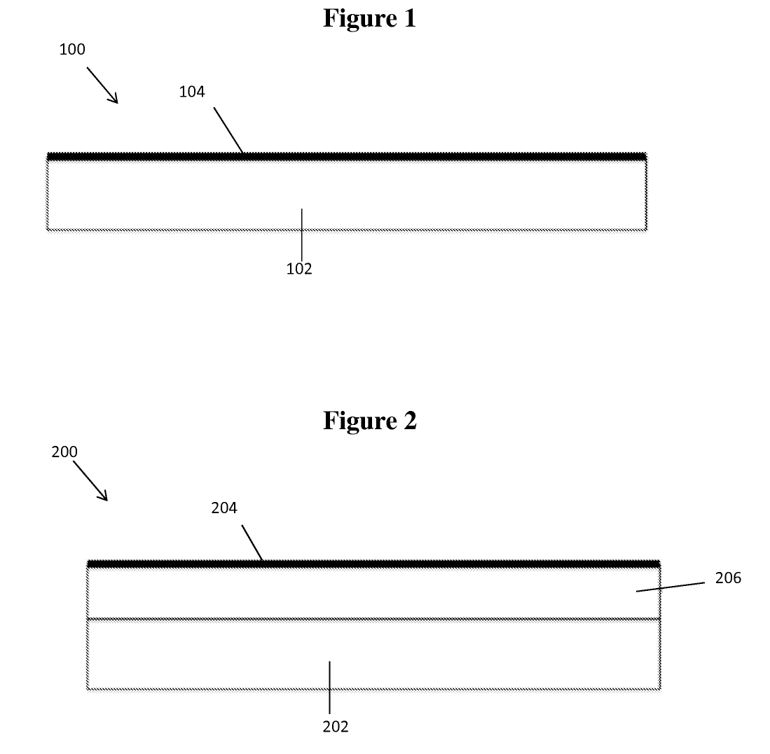

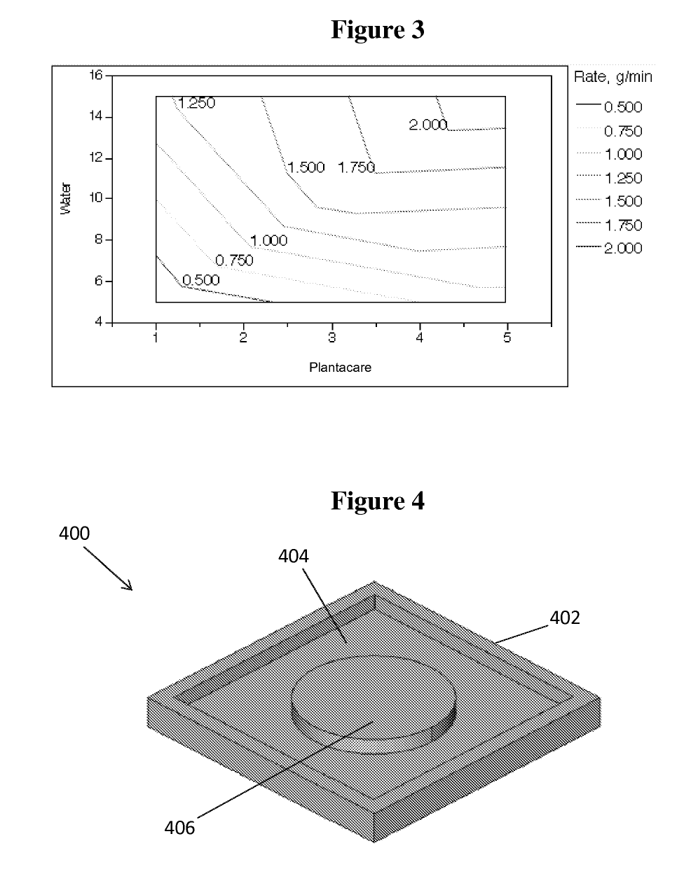

FIG. 1 illustrates an exemplary dressing 100. Dressing 100 contains honey-impregnated foam layer 102, which is suitable for complete or partial contact with a wound. Dressing 100 also contains backing sheet 104, which is located on the face of the dressing that is opposite to the face that is intended for contact with a wound.

FIG. 2 illustrates an exemplary dressing 200. Dressing 200 contains honey-impregnated foam layer 202, which is suitable for complete or partial contact with a wound. Dressing 200 contains backing sheet 204, which is located on the face of the dressing that is opposite to the face that is intended for contact with a wound. Dressing 200 further contains superabsorbent polymer layer 206, which is located between the honey-impregnated foam layer 202 and the backing sheet 204. The superabsorbent polymer layer 206 may be laminated to the honey-impregnated foam layer 202. The superabsorbent polymer layer 206 may allow for increased absorption of wound exudate and other fluids into the dressing relative to a similar dressing in which the superabsorbent polymer layer 206 is absent.

Foam dressings are widely known in the art and have been used extensively in wound treatment. Foams suitable for the compositions and methods described herein may be made of any suitable material including, without limitation, polyurethane (PU), polyvinyl alcohol (PVA), or silicone. The foam may be a hydrophilic foam, such as a hydrophilic PU foam, or a hydrophobic foam, such as a hydrophobic PU foam. The foam may be a PU laminate with a hydrofiber layer. The foam may be a lipido-colloid gel or a LiquaLock foam. The foam may be an open-cell reticulated foam. Exemplary foams include ALLEVYN® (Smith & Nephew, Inc.), BIATAIN® (Coloplast Corp.), DERMAFOAM® (DermaRite Industries), ELTA® Soft-touch foam dressing (SteadMed Medical), HYDROCELL® (Derma Sciences), HYDROFERA® (Hollister), MEPILEX® (Molnlycke Health Care), MPM EXCEL® Dressings (MPM Medical), OPTIFOAM® (Medline Industries), POLYDERM® (DeRoyal), POLYMEM® (Ferris Mfg. Corp.), TRIACT® wound dressing (Hollister), 3M TEGADERM® (3M Health Care), TIELLE® hydropolymer dressing (Systagenix), and VERSIVA XC® Gelling foam (ConvaTec).

The foam may be of any suitable shape or size, and the shape and size may be selected as appropriate for a particular wound. The foam may be square, rectangular, round, circular, or irregularly shaped. The foam may be suitably shaped for application to curved areas of the body, for instance, the heel of a foot or a sacral area. In some embodiments, the area of the foam may be about 1″×1″, 2″×2″, 3″×3″, 4″×4″, 5″×5″, 6″×6″, 8″×8″, 1″×2″, 2″×4″, 3″×6″, or 4″×8″. In some embodiments, the area of the foam may be at least about 1″×1″, 2″×2″, 3″×3″, 4″×4″, 5″×5″, 6″×6″, 8″×8″, 1″×2″, 2″×4″, 3″×6″, or 4″×8″. In some embodiments, the area of the foam may be no more than about 1″×1″, 2″×2″, 3″×3″, 4″×4″, 5″×5″, 6″×6″, 8″×8″, 1″×2″, 2″×4″, 3″×6″, or 4″×8″.

The foam may have about 30 to about 120 PPI, about 50 to about 100 PPI, about 60 to about 90 PPI, about 80 to about 110 PPI, or about 65 to about 85 PPI. The foam may have at least about 30 PPI, about 40 PPI, about 50 PPI, about 60 PPI, about 70 PPI, about 80 PPI, about 90 PPI, about 100 PPI, or about 110 PPI. The foam may have no more than about 40 PPI, about 50 PPI, about 60 PPI, about 70 PPI, about 80 PPI, about 90 PPI, about 100 PPI, about 110 PPI, or about 120 PPI.

The foams used in the compositions and methods described herein may be compressed prior to or following impregnation with honey. In some embodiments, the foam is subjected to a felting process. In the felting process, the foam is subjected to elevated temperature and/or pressure to modify its morphology. Following the application of elevated temperature and/or pressure, the foam does not fully relax back to its original morphology. The foam is thereby permanently or semi-permanently compressed to some degree. The felting process increases the effective density of the foam. The degree of compression controls the physical properties of the felt. In some instances, a foam that had a relatively uniform cell structure in all directions prior to felting will have cells that appear flattened or buckled following the felting process, and the cell structure will no longer be uniform in all directions. In a particular embodiment, the foam is a polyurethane foam that has undergone felting.

Felting may affect the fluid-to-foam interactions. For instance, a felted foam may have a different honey impregnation and/or release rate relative to the corresponding unfelted foam. The firmness of a felt can be described by the felting ratio, which is the ratio of the original thickness of the foam to the final thickness of the foam after the felting process is complete. Foams used in the compositions and methods described herein may have a felting ratio of about 2, 3, 4, 5, 6, 7, 8, 9, 10, 11, or 12. The foam may have a felting ratio of about 3-10, 4-8, 5-7, 2-6, 6-12, or 2-12. The foam may have a felting ratio of greater than about 2, 3, 4, 5, 6, 7, 8, 9, 10, 11, or 12.

The foams used in the compositions and methods described herein may be patterned, and this patterning may facilitate impregnation and/or release of the honey. The patterning of the foam may be regular or irregular. The foam may be patterned along the surface intended for contact with the wound. It may be patterned across part or all of the surface. The foam may also be patterned on surfaces other than those intended for contact with the wound. The patterning may take any form, such as grooves, grids, circles, honeycomb, channels, and squares. The pattern may form a series of positive and negative features on the surface of the foam, resulting one or more discrete wells or depressed areas. Such wells or depressed areas can be filled with honey. A border may be present as a positive feature of the pattern, and this border may facilitate containment of the honey and avoid or reduce leakage. The pattern may also contain positive features that are raised for contact with the wound. The positive features may facilitate release of honey onto the wound and/or may serve to draw out exudate from the wound. The positive and negative features of the pattern may be of uniform depth and/or height or of variable depth and/or height.

Patterning of the foam may be achieved by surface modification technology (SMT). SMT is a process by which a foam surface is altered. The alteration may include one or more alterations to pattern type, size, depth, spacing, and location of the foam. Exemplary patterns that may be made using SMT are shown in FIGS. 4-6.

FIG. 4 illustrates an exemplary patterned foam dressing 400. Border 402 contains the honey and prevents or reduces leakage. Depression 404 may be filled with honey. Center area 406 is raised for contact with the wound and to potentially draw out wound exudate. In a particular embodiment, the entire dressing 400 is approximately 2″×2″. Depression 404 is approximately ⅛″ deep and has an area of 1.75″×1.75″. Center area 406 has a diameter of approximately F. In another particular embodiment, the entire dressing 400 is approximately 3.2″×3.2″. Depression 404 is approximately ⅛″ deep and has an area of 2.75″×2.75″. Center area 406 has a diameter of approximately 0.5″.

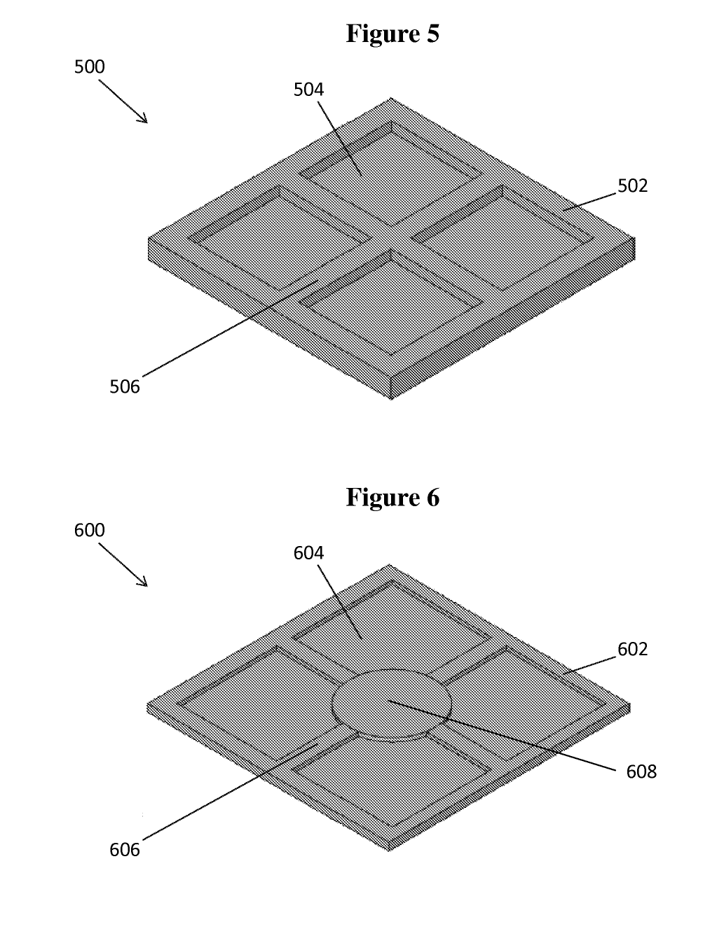

FIG. 5 illustrates an exemplary patterned foam dressing 500. Border 502 contains the honey and prevents or reduces leakage. Depressions 504 may be filled with honey. Internal grid lines 506 are raised for contact with the wound and to potentially draw out wound exudate. In a particular embodiment, the entire dressing 500 is approximately 3.2″×3.2″. Depressions 504 are approximately ⅛″ deep and have an area of 1.25″×1.25″. Internal grid lines 506 have a thickness of approximately 0.5″.

FIG. 6 illustrates an exemplary patterned foam dressing 600. Border 602 contains the honey and prevents or reduces leakage. Depressions 604 may be filled with honey. Internal grid lines 606 and central area 608 are raised for contact with the wound and to potentially draw out wound exudate. In a particular embodiment, the entire dressing 600 is approximately 8″×8″. Depressions 604 are approximately ⅛″ deep and have an area of 3.25″×3.25″. Internal grid lines 606 have a thickness of approximately 0.5″, and central area 608 has an approximate diameter of 2.75″.

The compositions described herein contain honey. Any type of honey may be used in the compositions. The honey may be from a single source or from multiple sources. The honey may have antimicrobial or bacteriostatic properties. Typically, the honey is medical grade honey. The honey may have peroxide activity or non-peroxide activity or both. In some embodiments, the honey is derived from Leptospermum, such as Leptospermum scoparium (manuka honey) or Leptospermum polygalifollum (jellybush honey).

The compositions described herein may contain a surfactant. The surfactant may be an ionic (e.g., cationic or anionic) surfactant or a non-ionic surfactant. The surfactant may be a carbohydrate-based surfactant, such as a glucoside surfactant. The surfactant may be an alkyl glucoside or an alkyl polyglucoside. The surfactant may be a fatty alcohol glucoside or a fatty alcohol polyglucoside. The surfactant may be a coco glucoside or a cocopolyglucoside. In particular embodiments, the surfactant is lauryl glucoside, decyl glucoside, or caprylyl/capryl glucoside. In other embodiments, the surfactant is sucrose acrylate. In yet other embodiments, the surfactant is PEG-n methyl glucose acrylate.

The foam dressings described herein may contain a therapeutically effective amount of honey. The foam dressing may be impregnated with honey at a level of at least about 0.5, 0.6, 0.7, 0.8, 0.9, 1.0, 1.1, 1.2, 1.3, 1.4, 1.5, 1.6, 1.7, 1.8, 1.9, or 2.0 grams per square inch. In a particular embodiment the foam dressing is impregnated with honey at a level of at least about 1.6 grams per square inch. The foam dressing may be impregnated with honey at a level of about 0.5 to about 2.0 grams per square inch. The foam dressing may be impregnated with honey at a level of about 1.0 to about 2.0, about 0.9 to about 1.6, about 1.2 to about 1.8 or about 1.4 to about 2.0 grams per square inch. The level of impregnation may be uniform across the entire foam dressing, or the dressing may contain the desired level of impregnation over a portion the foam dressing. In some embodiments, the level of impregnation is an average level of impregnation over the entire foam dressing or an average level of impregnation over a portion of the foam dressing.

In some embodiments, the foam dressing absorbs saline in an amount that is greater than about 5 times, greater than about 6 times, greater than about 7 times, greater than about 8 times, greater than about 9 times, or greater than 10 times the weight of the foam. Saline absorption can be measured using methods known in the art including, without limitation, according to the methods described in “Test methods for primary wound dressings. Aspects of absorbency.” (BS EN 13726-1-2002, British Standards Institute).

The compositions described herein may contain a tackiness-reducing agent. Suitable tackiness-reducing agents are known in the art and are commercially available. Such agents render a material easier to handle by forming a reduced-tackiness or non-tacky layer on the surface of the impregnated material, thereby forming a barrier to the underlying tacky material. Suitable tackiness-reducing agents include, without limitation, hydrocollids, polyacrylate superabsorbent polymers, alginates, beta glucan, gums (e.g., xanthan gum, acacia gum), uronic acids (e.g., guluronic acid, manuronic acid), starches, and other plant exudates. The tackiness-reducing agent may be impregnated into the foam dressing, or it may be present on the surface of the dressing. It may be used in the form of a powder or as granules. The tackiness-reducing agent may be applied to the foam dressing prior to, concurrently with, or subsequent to application of the honey to the dressing. In a particular embodiment, the tackiness-reducing agent is carboxymethylcellulose (CMC). The CMC may be of low, medium, or high molecular weight.

The compositions described herein may contain a backing sheet. The backing sheet may serve a variety of purposes. The backing sheet may provide a means of adding an adhesive component to the composition, provide a barrier to prevent leakage of honey, wound exudate, or other liquids from the dressing, act as an intermediate between the foam and further backing layers, or act as a protective covering over the foam dressing. Typically, the backing sheet is applied to the face of the dressing that is opposite to the face that is intended for contact with a wound. The backing sheet may cover a portion of the foam dressing, it may be co-extensive with the foam dressing or it may extend beyond the foam dressing. The backing sheet may be adhesive, such as to adhere the dressing to the surface of the skin surrounding a round. The backing sheet may be non-adhesive. The backing sheet may be partially adhesive and partially non-adhesive. The backing sheet may be porous or substantially non-porous. The backing sheet may contain a single layer or multiple layers of one or more suitable materials. The backing sheet may be applied to the foam dressing prior to or subsequent to impregnation with honey. The backing sheet may be applied to the foam dressing using any means known in the art.

A cover sheet may be applied to cover the backing sheet and/or the foam dressing. The cover sheet may be co-extensive with the backing sheet and/or the foam dressing, or it may extend beyond the backing sheet and/or the foam dressing. The cover sheet may be used to reduce tackiness in addition to or in place of a tackiness-reducing agent. The cover sheet may be temporarily adhered to the backing sheet with an adhesive. The cover sheet may be configured to be peeled off by the user prior to applying the composition. Peeling off the cover sheet may expose the foam and/or the adhesive applied to the backing sheet. The cover sheet can be any suitable material, including, for example, a polymer film liner. The cover sheet may be applied to the backing sheet and/or the foam dressing using any means known in the art.

The dressings described herein may optionally contain other components including, without limitation, active pharmaceutical agents such as fungicides or antibacterial agents, fragrances, skin-conditioning agents, essential oils, or any agents that promote wound healing.

The foam dressing may be sterile. Sterilization can be achieved by methods known in the art, such as gamma irradiation. Typically, a sterilization method is employed that does not substantially interfere with the antimicrobial or bacteriostatic properties of the honey. Additionally, the compositions may be contained within a sterile package. Suitable packaging materials and methods are known in the art.

In one aspect, the compositions described herein may contain a super absorbent polymer (SAP) layer. The SAP layer may be laminated to the foam. One example of this configuration is the commercially available XTRASORB® (Derma Sciences, Inc.). Such a configuration may provide for all of the advantages of a honey-impregnated foam that lacks the SAP, while also providing increased capacity to remove moisture (e.g., wound exudate and other fluids) from the wound. The SAP may be laminated to the foam prior to or subsequent to impregnation of the foam with honey. The SAP-foam laminate may contain a backing sheet applied to the surface of the SAP (i.e., the surface opposite the face of the foam intended for contact with the wound).

### Methods of Preparation

Provided herein are methods of preparing the compositions described herein. Impregnation of honey into a foam dressing can be achieved by direct coat, that is, by applying a mixture of honey and water to the surface of the foam dressing. The mixture applied to the surface of the foam dressing may additionally contain a carbohydrate-based surfactant. Addition of a carbohydrate-based surfactant may improve the rate and/or degree of penetration of the honey into the foam dressing. Using a mixture of honey, surfactant, and water, impregnation of honey into the foam can be achieved at a rate of at least about 0.5, 0.75, 1.0, 1.25, 1.5, 1.75, 2.0, 2.5, or 3.0 grams/min using, for example, a manual process with a brayer.

Impregnation of honey into the foam can also be achieved by transfer coating. In this process, the honey is applied to a release liner that is then pressed into contact with the foam. The properties of the release liner facilitate transfer of honey to the foam. In some embodiments, the release liner contains polyethylene terephthalate (PET). The release liner may be hydrophobic, or may be coated with a hydrophobic substance. Exemplary materials for the release liner or liner coating are silicon-based, fluorocarbon-based, or polyolefin-based materials. Exemplary release liners include PET liners, TECHNICOTE® L-3 silicone coated PET liner (5Mil) (Technicote, Miamisburg, Ohio); CHEMFAB® PTFE coated belts and fabrics (Quadrant Plastic Composites, Northville, Mich.); and FLEXcon-DigiPRO® DGPPW White Polyolefin Roll Liner (FLEXcon, Spencer, Mass.).

The mixture applied to the surface of the foam dressing may contain at least about 20, 30, 40, 50, 60, 70, 80, 90, or 95% honey. The mixture applied to the surface of the foam dressing may about 20 to about 80, about 30 to about 70, about 40 to about 60, about 20 to about 60, about 40 to about 80, about 60 to about 90, or about 70 to about 95% honey.

The mixture applied to the surface of the foam dressing may contain at least about 5, 10, 20, 30, 40, or 50% water. The mixture applied to the surface of the foam dressing may contain at no more than about 60, 50, 40, 30, 20, or 10% water. The mixture applied to the surface of the foam dressing may contain about 10 to about 60, about 20 to about 50, about 30 to about 50, about 10 to about 40, about 10 to about 20, about 5 to about 15, or about 40 to about 60% water.

The mixture applied to the surface of the foam dressing may contain at least about 0.01, 0.1, 0.5, 1.0, 2.0, or 5.0% surfactant. The mixture applied to the surface of the foam dressing may contain no more than about 0.1, 0.5, 1.0, 2.0, or 5.0% surfactant. The mixture applied to the surface of the foam dressing may contain about 0.01 to about 5.0, about 0.1 to about 2.0, about 0.5 to about 1.0, about 0.5 to about 2.0 or about 1.0 to about 2.0% surfactant.

In some embodiments, the mixture applied to the surface of the foam dressing contains about 80 to about 94% honey, about 5 to about 15% water, and 1 to about 5% surfactant. In a particular embodiment, the mixture is about 86% honey, about 10% water, and about 3% surfactant. In another particular embodiment, the mixture is about 80% honey, about 15% water, and about 5% surfactant. In another particular embodiment, the mixture is about 75% honey, about 24% water, and about 1% surfactant.

Conditions may be modified to optimize the rate and/or degree of penetration of honey into the foam dressing. The methods described herein may be carried out at room temperature or under mild heat, such as in a temperature range of 35 to 45° C. (e.g., 38 to 42° C.). The temperature is typically maintained in a suitable range for maintaining the antimicrobial properties of the honey. Mechanical means such as pressure and/or vibration may also be applied to the foam to facilitate penetration of the honey.

Vacuum may be used to facilitate impregnation of honey into the foam. Vacuum may be applied to the surface of the foam opposite the surface on which the honey or honey mixture is applied to facilitate the process of drawing the honey into the foam. A vacuum may be applied until the foam is partially or completely saturated with the honey or honey mixture. Use of a vacuum may be particularly desirable for impregnating honey into a foam that lacks a backing sheet, particularly a foam that lacks an impermeable backing sheet. A backing sheet may be applied to the foam subsequent to impregnation of the foam with honey.

In some embodiments, the vacuum is applied until the honey has penetrated only a portion of the foam such that there is a greater honey content in the portion of the foam closest to the face intended for contact with the wound relative to the portion of the foam closest to the face intended to be opposite the wound. This differential or gradient of honey across the foam may allow for the wound-healing benefits of the honey in the foam dressing while providing an unsaturated portion of the foam that has additional capacity to absorb wound exudate and other fluids.

After impregnation, the composition may be dried to reduce the total water content in the dressing using methods known in the art including, without limitation, heat, high frequency ultraviolet light, or infrared light.

The foam may be cut to an appropriate size for application to a wound prior to impregnation, or a larger piece of foam may be impregnated with honey and then cut an appropriate size for application to a wound. An automated roller may be used to apply the honey mixture to the foam.

### Methods of Use

Compositions described herein may be used in treatment of wounds in an individual including, without limitation, skin ulcers, leg ulcers, diabetic foot ulcers, pressure ulcers, sutured wounds, skin grafts, burns, wounds requiring negative pressure wound therapy, surgical wounds, oral wounds, and exuding wounds. The wound may be the result of an acute condition or the result of a chronic condition.

A single foam dressing may be applied for a period of several hours, several days, or several weeks. In some embodiments, the foam dressing is changed every two- to five-days during treatment. In some embodiments, the foam dressing is changed every three- to four-days during treatment. The foam dressing may be held in place by an adhesive backing sheet integrally attached to the foam, or it may be held in place by a secondary dressing, tape, wrap, or net.

The compositions described herein may be used in combination with other forms of treatment. Such other forms of treatment may be primary forms of treatment for the wound itself, or they may be forms of treatment of an underlying condition associated with the wound. Such other forms of treatment include, without limitation, other pharmaceutical agents, which may be administered by any appropriate means (e.g., oral, topical, intravenous), surgical methods, suturing, skin or tissue grafting, hyperbaric oxygen therapy, nutritional management, physical therapy, dialysis, nuclear medicine, draining techniques, and herbal remedies.

### EXAMPLES

### Example 1

Approximately 10 g of medical grade honey was applied to the surface of a 4″×4″ HYDROCELL® foam dressing (Derma Sciences, Inc.). The honey remained on the surface of the dressing and did not substantially penetrate the dressing material.

### Example 2

Approximately 10 g of medical grade honey was applied to the surface of a 4″×4″ HYDROCELL® foam dressing (Derma Sciences, Inc.) at room temperature. A brayer was used to apply mild to moderate pressure on the honey. The honey spread unevenly on the surface of the dressing material. The honey did not substantially penetrate the dressing material. Increasing the temperature to 38-42° C. did not improve penetration of the honey into the dressing material. Vibration did not improve penetration of the honey into the dressing material. Roller hardness and wettability (i.e., hydrophobic or hydrophilic) had little to no effect on penetration of the honey into the dressing material.

### Example 3

Approximately 10 g of medical grade honey was applied to the surface of a 4″×4″ HYDROCELL® foam dressing (Derma Sciences, Inc.) and sealed in a foil pouch to prevent moisture loss. After 24 hours at 40° C., no significant amount of honey had penetrated the dressing material. Applying light to moderate pressure did not improve penetration.

### Example 4

Using a transfer pipet, a drop of 50% solution of medical grade honey in water was applied to the surface of a 4″×4″ HYDROCELL® foam dressing (Derma Sciences, Inc.). The honey solution slowly penetrated the foam within 5-10 minutes.

### Example 5

A solution containing 50% honey, 49% water, and 1% caprylyl/capryl glucoside surfactant (PLANTACARE® 810 UP, BASF) was applied to the surface of a 4″×4″ HYDROCELL® foam dressing (Derma Sciences, Inc.). The solution immediately penetrated the dressing material.

### Example 6

A factorial experiment plan with center point was used to screen the following:

1 to 5% PLANTACARE® 810 UP

5 to 15% water

Sufficient honey to achieve 100%

For each formulation tested, the time required to press in about 2 to 2.5 g of honey solution into a 2″×2″ section of dressing material using a brayer was measured. The brayer was manually vibrated up and down slightly during rolling. The temperature was maintained at 38-42° C. A contour plot showing the rate of impregnation of the honey solution across the range of variation in water and PLANTACARE percentages is shown in FIG. 1.

### Example 7

Surfactants were screened for their ability to facilitate impregnation of honey into a HYDROCELL foam dressing. The surfactants were tested at 1% in a 75%/24% honey/water formulation at 38-42° C. The surfactants tested were:

Polysorbate 80

Sodium lauryl sulfate

Tyloxapol

caprylyl/capryl glucoside

Of the surfactants tested, only caprylyl/capryl glucoside significantly increased the rate and extent of impregnation of honey into the foam dressing, compared to a honey/water formulation lacking surfactant.

### Example 8

Two different foams (Z-4 and HP-1 from FXI) were run on a pilot coating line using approximately 35 ft. rolls of foam run at 3 ft/min. A transfer process was used to apply the honey to the foam. Honey was poured onto a silicon-based release liner that was then applied the surface of the foam. At the end of the coating line, the foam and release liners passed through a lamination (i.e., application of pressure) station. The rolls were 4″ wide. Z-4 had about 65 PPI and was more permeable than the HP-1, which had about 80 PPI. Application of neat honey, honey formulated with surfactant only, and honey formulated with surfactant and water were tested for samples of each of the foam materials.

The Z-4 foam was able to absorb 93-96% of the applied honey or honey formulation during the coating process. The HP-1 foam was able to absorb 93% of a honey formulation with 1% PLANTACARE and 5% water during the coating process. Very little of the honey formulation was left adhering to the surface of the foam. The HP-1 foam was not able to absorb or adhere more than about 75% of neat honey or honey with 1% PLANTACARE during the coating process. A significant amount of honey remained on the surface of the foam. Thus, the water/honey combination was important for the HP-1 foam.

### Example 9

Two different polyurethane foams (FXI) were tested for their ability to absorb and release honey and for their ability to absorb saline after impregnation with honey. Foams 1 and 2 were prepared as 4 inch by 4 inch squares. Foams 1 and 2 were prepared with no patterning or were patterned with a donut-shaped depression, which was created using surface modification technology (SMT). The donut shapes had an inner diameter of 1.5 inches and outer diameter of 3.5 inches. To each of the foam samples was applied a honey formulation containing 94% honey, 5% water, and 1% PLANTACARE. The foams were allowed to equilibrate for 48 hours following application of the honey formulation. The amount of honey that became impregnated into the foam is indicated in Tables 1 and 2.

The foam samples were then placed in saline solution at 37° C. for 4 hours. The foam samples were place honey-side down in the saline solution. The saline solution contained 8.298 g of NaCl and 0.368 g of CaCl monohydrate per liter of purified water (in accordance with BS EN 13726-1-2002, British Standards Institute) and was equilibrated to 37° C. before the test began. 360 mL of saline solution was used per sample. The amount of honey released and saline absorbed is indicated in Tables 1 and 2. Tables 1 and 2 represent the same procedure performed at different times and on different individual samples.

Measurements were determined as follows: naked foam samples were weighed before adding honey (average foam weight). The amount of honey impregnated into the foam was determined by subtracting the average foam weight from the honey-impregnated foam. After incubation in the saline solution, the extracted foam weight was measured, which corresponds to the weight of foam and absorbed saline minus the weight of honey released.

The foam samples were then dried by storing the samples at 40° C. for 24 hours and weighed (dried foam weight). The amount of saline absorbed was calculated as the extracted foam weight minus the dried foam weight. The percentage of honey released was calculated by subtracting the average foam weight from the dried foam weight, and then dividing the difference by the amount of honey impregnated in the foam (% of honey released=100*(Dried foam weight−average foam weight)/(honey impregnated)).

TABLE 1

| | Average | Honey | Honey | Saline | Saline |
|---|---|---|---|---|---|
| | Foam | Impregnated, | Released, | Absorbed, | Absorbed, |
| Foam | Weight, g | g | % | g/in² | g/g |
| Foam 1, | 3.1 | 25.6 | 89 | 1.8 | 9.5 |
| patterned | | | | | |
| Foam 1, | 4.0 | 18.3 | 86 | 2.8 | 11.1 |
| no | | | | | |
| patterning | | | | | |
| Foam 2, | 3.1 | 24.9 | 88 | 1.9 | 10.0 |
| patterned | | | | | |
| Foam 2, | 4.0 | 19.6 | 89 | 2.4 | 9.9 |
| no | | | | | |
| patterning | | | | | |

TABLE 2

| | Average | | | | |
|---|---|---|---|---|---|
| | Foam | Honey | Honey | Saline | Saline |
| | Weight, | Impregnated, | Released, | Absorbed, | Absorbed, |
| Foam | g | g | % | g/in² | g/g |
| Foam 1, | 3.1 | 24.6 | 91 | 1.9 | 9.3 |
| patterned | | | | | |
| Foam 1, no | 4.0 | 20.6 | 92 | 2.8 | 10.7 |
| patterning | | | | | |
| Foam 2, | 3.1 | 24.8 | 89 | 1.9 | 9.8 |
| patterned | | | | | |
| Foam 2, no | 4.0 | 20.7 | 90 | 2.4 | 9.7 |
| patterning | | | | | |

### Example 10

Three different honey formulations were tested for their ability to be absorbed into a felted polyurethane foam using two different release liners. The three honey formulations tested were: 1) 5% Water, 94% Medihoney, 1% and Plantacare; 2) 3% Water, 96% Medihoney, and 1% Plantacare; and 3) 1% Water, 98% Medihoney, and 1% Plantacare. The release liners tested were a 5 Mil TECHNICOTE® L-3 silicone coated PET liner and a 5 Mil plain PET liner. The foam used was 16″ wide, with linear footage coated with honey formulation as indicated in Table 3.

Honey formulation was applied to the liner using a knife over roll coating technique. The knife blade was a comma bar. The distance between the knife blade and the liner (gap setting) was varied between runs to allow for the application of different amounts of honey formulation, as determined by coat weight (see Table 3). The coat weight of the applied honey formulation was then assessed by measuring the difference in weight between the naked release liner and the release liner after honey formulation was applied.

The foam was then placed into contact with the honey formulation, allowing the honey formulation to permeate the foam.

The release liner and foam were then placed in an oven to allow the foam to absorb the honey formulation. Various oven conditions were tested, including support and curing temperature (see Table 3). The types of support tested included an unsupported web, supported web, heat and airflow, and airflow only.

TABLE 3

| | | Oven | Line | Gap | Coat | | |
|---|---|---|---|---|---|---|---|
| Roll | Honey | Temp | Speed | Setting | Weight | Linear | Oven |
| # | Formulation | (° F.) | (ft/min) | (mil) | (g/m²) | Footage | Support |
| 1 | 5% H₂O | 71 | 3 | 20 | 390 | 25 | Unsupported |
| | 94% Medihoney | | | | | | in oven |
| | 1% Plantacare | | | | | | |
| 2 | 5% H₂O | 71 | 3 | 18 | 305 | 35 | Unsupported |
| | 94% Medihoney | | | | | | in oven |
| | 1% Plantacare | | | | | | |
| 3 | 5% H₂O | 71 | 3 | 70 | 1522 | 37 | Supported |
| | 94% Medihoney | | | | | | on boards |
| | 1% Plantacare | | | | | | |
| 4 | 1% H₂O | 130 | 10 | 70 | 1565 | 40 | Supported |
| | 98% Medihoney | | | | | | by airflow |
| | 1% Plantacare | | | | | | |
| 5 | 1% H₂O | 85 | 3 | 18 | 307 | 40 | Supported |
| | 98% Medihoney | | | | | | by airflow |
| | 1% Plantacare | | | | | | |
| 6 | 3% H₂O | 80 | 3 | 110 | 2721 | 40 | Supported |
| | 96% Medihoney | | | | | | by airflow |
| | 1% Plantacare | | | | | | |
| 7 | 1% H₂O | 80 | 3 | 18 | 333 | 20 | Supported |
| | 98% Medihoney | | | | | | by airflow |
| | 1% Plantacare | | | | | | |

After the foam passed through the oven, passed through rollers applying light compression, and collecting the foam and release liner onto a roll, the release liner was removed and the amount of honey formulation absorbed by the foam was determined. The percentage of honey formulation absorbed by the foam was calculated by comparing the weight of honey formulation absorbed by the foam to the amount of honey applied to the release liner (Table 4 and Table 5).

TABLE 4

| | | Percentage | Percentage |
|---|---|---|---|
| | | of honey | of honey |
| | | formulation | formulation |
| | | absorbed | absorbed |
| | | <10 min post | 30 min post |
| Honey Formulation | Release Liner | coating | coating |
| 5% H₂O | Silicone-coated | >85% | >92% |
| 94% Medihoney | PET | | |
| 1% Plantacare | | | |

TABLE 5

| | | | Percentage of | Percentage of |
|---|---|---|---|---|
| | | Percentage of | honey | honey |
| | | honey | formulation | formulation |
| | | formulation | absorbed 24 | absorbed 72 |
| Honey | Release | absorbed 10 min | hours post | hours post |
| Formulation | Liner | post coating | coating | coating |
| 1% H₂O | Silicone- | >80% | >85% | >85% |
| 98% Medihoney | coated PET | | | |
| 1% Plantacare | | | | |
| 1% H₂O | Plain PET | >80% | >80% | >80% |
| 98% Medihoney | | | | |
| 1% Plantacare | | | | |

### Example 11

The absorbency of three different polyurethane foams with varying degrees of felting (Foam 1, Foam 2, and Foam 3) were tested. All tests were performed similarly to the method described in “Test methods for primary wound dressings. Aspects of absorbency.” (BS EN 13726-1-2002). A saline solution was prepared with 8.298 g of NaCl and 0.368 g of CaCl monohydrate per liter of purified water. 1 inch×1 inch square samples of foam were weighed and placed in petri dishes. The saline solution was then added to the petri dish at an amount equal to 120 times the weight of the foam. The foam samples were saturated in the saline solution for two hours at 37° C. and then weighed again. The results are shown as a ratio of the weight of saline absorbed per weight of foam. The results are shown in Table 6.

TABLE 6

| | Weight of saline absorbed per | | |
|---|---|---|---|
| | weight of foam | | |
| Felting | Foam 1 | Foam 2 | Foam 3 |
| No felting | 30.1 | 31.0 | 24.6 |
| Moderate felting | 14.8 | 13.6 | 10.6 |
| High felting | 8.8 | 8.8 | 8.0 |

Modifications may be made to the foregoing without departing from the basic aspects of the methods and compositions provided herein. Although the compositions and methods have been described in substantial detail with reference to one or more specific embodiments, those of skill in the art will recognize that changes may be made to the embodiments specifically disclosed in this application, yet these modifications and improvements are within the scope and spirit of the methods and compositions provided herein.

All documents, including patents, patent application and publications cited herein, including all documents cited therein, tables, and drawings, are hereby expressly incorporated by reference in their entirety for all purposes.

While the methods and compositions have been described in detail with reference to certain Exemplary aspects thereof, it will be understood that modifications and variations are within the spirit and scope of that which is described and claimed.

1. A composition for treating wounds, comprising foam impregnated with honey and a surfactant.

2. The composition of claim 1, wherein the surfactant is a carbohydrate-based surfactant.

3. The composition of any one of claims 1-2, wherein the surfactant is a glucoside surfactant.

4. The composition of any one of claims 1-3, wherein the honey has antimicrobial or bacteriostatic properties.

5. The composition of any one of claims 1-4, wherein the honey has non-peroxide antimicrobial or bacteriostatic properties.

6. The composition of any one of claims 1-5, further comprising a tackiness-reducing agent.

7. The composition of claim 6, wherein the tackiness-reducing agent is carboxymethylcellulose (CMC).

8. The composition of any one of claims 1-7, further comprising a backing sheet.

9. The composition of claim 8, wherein the backing sheet is adhesive.

10. The composition of any one of claims 1-9, wherein the foam has about 40 to about 100 PPI.

11. The composition of any one of claims 1-10, wherein the foam is a polyurethane foam.

12. The composition of any one of claims 1-11, further comprising beta-glucan.

13. The composition of any one of claims 1-12, wherein the composition is impregnated with a therapeutically effective amount of the honey.

14. The composition of any one of claims 1-13, wherein the composition further comprises a super-absorbent polymer laminated to the foam.

15. The composition of any one of claims 1-14, wherein the composition further comprises a peelable cover sheet adhered to the dressing.

16. The composition of any one of claims 1-15, wherein the composition is sterile.

17. A composition for treating wounds, comprising foam impregnated with honey at a level of at least about 0.5 grams per square inch.

18. The composition of claim 17, wherein the foam dressing is impregnated with honey at a level of at least about 0.9 grams per square inch.

19. The composition of any one of claims 17-18, wherein the foam dressing is impregnated with honey at a level of at least about 1.6 grams per square inch.

20. The composition of one of claims 17-19, further comprising a surfactant.

21. The composition of claim 20, wherein the surfactant is a carbohydrate-based surfactant.

22. The composition of claim 20, wherein the surfactant is a glucoside surfactant.

23. The composition of one of claims 17-22, wherein the honey has antimicrobial or bacteriostatic properties.

24. The composition of one of claims 17-23, wherein the honey has non-peroxide antimicrobial or bacteriostatic properties.

25. The composition of one of claims 17-24, further comprising a tackiness-reducing agent.

26. The composition of claim 25, wherein the tackiness-reducing agent is carboxymethylcellulose (CMC).

27. The composition of one of claims 17-26, further comprising a backing sheet.

28. The composition of claim 27, wherein the backing sheet is adhesive.

29. The composition of one of claims 17-28, wherein the foam has about 40 to about 100 pores per inch (PPI).

30. The composition of one of claims 17-29, wherein the foam is a polyurethane foam.

31. The composition of one of claims 17-30, further comprising beta-glucan.

32. The composition of one of claims 17-31, wherein the composition further comprises a super-absorbent polymer laminated to the foam.

33. The composition of any one of claims 17-32, wherein the composition further comprises a peelable cover sheet adhered to the dressing.

34. The composition of any one of claims 17-33, wherein the composition is sterile.

35. A method of making a wound dressing, comprising:
a) mixing honey, water, and a surfactant; and
b) applying the mixture of step a) to the surface of a piece of foam.

36. The method of claim 35, wherein the mixture of step a) comprises at least about 50% honey.

37. The method of any one of claims 35-36, wherein the mixture of step a) comprises less than about 50% water.

38. The method of any one of claims 35-37, wherein the mixture of step a) comprises about 0.5 to about 5% surfactant.

39. The method of any one of claims 35-38, wherein the mixture of step a) comprises about 80 to about 94% honey, about 5 to about 15% water, and about 1 to about 5% surfactant.

40. The method of any one of claims 35-39, wherein one or both of steps a) and b) are carried out at room temperature.

41. The method of any one of claims 35-40, wherein the honey is at a temperature of between about 35 and about 45° C. at some time during the process.

42. The method of any one of claims 35-41, further comprising drying the foam after step b) to reduce the water content.

43. The method of any one of claims 35-42, further comprising applying a tackiness-reducing agent to the foam after step b).

44. The method of claim 43, wherein the tackiness-reducing agent is carboxymethylcellulose (CMC).

45. A method of treating a wound in an individual, comprising applying the composition of any one of claims 1-16 thereto.

46. The method of claim 45, wherein the wound is selected from the group consisting of a skin ulcer, a leg ulcer, a diabetic foot ulcer, a pressure ulcer, a sutured wound, a skin graft, a burn, a wound requiring negative pressure wound therapy, a surgical wound, an oral wound, and an exuding wound.

47. A method of treating a wound in an individual, comprising applying the composition of any one of claims 17-34 thereto.

48. The method of claim 47, wherein the wound is selected from the group consisting of a skin ulcer, a leg ulcer, a diabetic foot ulcer, a pressure ulcer, a sutured wound, a skin graft, a burn, a wound requiring negative pressure wound therapy, a surgical wound, an oral wound, and an exuding wound.

49. The process of any one of claims 1-16, wherein the foam is a felted foam.

50. The process of any one of claims 1-16, wherein the foam is a patterned foam.

51. The process of any one of claims 17-34, wherein the foam is a felted foam.

52. The process of any one of claims 17-34, wherein the foam is a patterned foam.
